(19) United States
(12) Patent Application Publication
(10) Pub. No.: US 2017/0184577 A1
(43) Pub. Date: Jun. 29, 2017

(54) SENSING SENSOR AND SENSING DEVICE

(71) Applicant: NIHON DEMPA KOGYO CO., LTD., Tokyo (JP)

(72) Inventor: Hiroyuki KUKITA, Saitama (JP)

(73) Assignee: NIHON DEMPA KOGYO CO., LTD., Tokyo (JP)

(21) Appl. No.: 15/389,452

(22) Filed: Dec. 23, 2016

(30) Foreign Application Priority Data
Dec. 25, 2015 (JP) 2015-255021

(51) Int. Cl. G01N 33/543; G01N 33/487

(52) U.S. Cl. CPC G01N 33/5438; G01N 33/48792; G01N 33/54386

(57) ABSTRACT

A sensing sensor includes a wiring board, a piezoelectric resonator, a channel forming member, a case body, and a regulating portion. The case body houses the wiring board, the piezoelectric resonator, and the channel forming member. The case body includes a window faced to a region including the output terminal on the another surface side of the wiring board and an injection port supplying a supply liquid to the one end side of the channel. The regulating portion is disposed in the case body. The regulating portion regulates the wiring board from the one surface side when the wiring board is pressed from the another surface side. The piezoelectric resonator is pressed against the surrounding portion by the output terminal being pressed from the another surface side.

### CROSS-REFERENCE TO RELATED APPLICATIONS

This application is based on and claims priority under 35 U.S.C. §119 to Japanese Patent Application No. 2015-255021, filed on Dec. 25, 2015, the entire content of which is incorporated herein by reference.

### TECHNICAL FIELD

This disclosure relates to a sensing sensor and a sensing device that sense a sensing object included in a sample solution on the basis of an oscillation frequency of a piezoelectric resonator.

### DESCRIPTION OF THE RELATED ART

In a clinical field, a simple method called Point of Care Testing (POCT), which is typified by a self-monitoring of blood glucose level, for example, is spreading. As an example of this method, Japanese Unexamined Patent Application Publication No. 2012-145566 describes a sensing sensor using Quartz Crystal Microbalance (QCM) and discloses a structure that forms a housing space for a sample solution between an upper-side case and a crystal resonator by placing the crystal resonator on a wiring board and engaging the upper-side case and a lower-side case with each other. As a sensitivity enhancing technique of QCM, there are twin sensors that perform a differential measurement. This technique forms two electrodes symmetrically with respect to a channel on one crystal such that the sample solution simultaneously and similarly flows with respect to the electrodes to cancel an ambient external noise.

With a sensing sensor, narrowing a height of a channel to improve a contact ratio of a sensing object to a surface of a crystal resonator is desired. Japanese Unexamined Patent Application Publication No. 2011-27716 describes a structure that sets a height of a channel to 0.2 mm. However, shape variations of a fitted cover may collapse the channel or break the crystal resonator when a channel forming member is pressed. A method that reduces a pressure from the cover that is fitted by narrowing a depressed portion of a sheet may be possible, but a sample solution may leak outside the sensor. Japanese Unexamined Patent Application Publication No. 2005-201912 describes the following structure. Electrode portions to absorb a sensing object are disposed near a center on a top surface side of a crystal resonator placed on a base. Pressing spring terminals from the above to terminal portions extended to peripheral edges of the electrode portions on the top surface side sandwiches the crystal resonator. However, this does not solve a problem of this disclosure.

Furthermore, recently, there has been known a sensing sensor that performs a differential measurement with respective electrode pairs using two pairs of electrodes as described in Japanese Unexamined Patent Application Publication No. 2004-69661. Such a sensing sensor has a problem of increased size of the sensing sensor due to the increased number of terminal portions to output a frequency from the sensing sensor.

A need thus exists for a sensing sensor and a sensing device which are not susceptible to the drawback mentioned above.

### SUMMARY

According to this disclosure, there is provided a sensing sensor that includes a wiring board, a piezoelectric resonator, a channel forming member, a case body, and a regulating portion.

The wiring board includes a depressed portion formed on one surface side, an output terminal formed on another surface side, and a conductive path connected to the output terminal and extended to the one surface side. The piezoelectric resonator is configured by disposing an excitation electrode electrically connected to the conductive path in a piezoelectric piece. The piezoelectric resonator is formed with an adsorbing layer adsorbing a sensing object in a sample solution on the one surface side. The piezoelectric resonator is secured to the wiring board in a state where the depressed portion is covered such that a vibrating region is opposed to the depressed portion. The channel forming member is disposed to cover a region on the one surface side of the wiring board including the piezoelectric resonator. The channel forming member includes a surrounding portion forming a channel that passes a supply liquid from one end side to another end side on the one surface side of the piezoelectric resonator between the piezoelectric resonator and the channel forming member.

The case body houses the wiring board, the piezoelectric resonator, and the channel forming member. The case body includes a window faced to a region including the output terminal on the another surface side of the wiring board and an injection port supplying a supply liquid to the one end side of the channel. The regulating portion is disposed in the case body. The regulating portion regulates the wiring board from the one surface side when the wiring board is pressed from the another surface side. The piezoelectric resonator is pressed against the surrounding portion by the output terminal being pressed from the another surface side.

### BRIEF DESCRIPTION OF THE DRAWINGS

The foregoing and additional features and characteristics of this disclosure will become more apparent from the following detailed description considered with reference to the accompanying drawings, wherein:

FIG. 1 is a perspective view of a sensing device using a sensing sensor according to this disclosure;

FIG. 2 is an exploded perspective view of the sensing sensor;

FIG. 3 is an exploded perspective view illustrating a top surface side of respective portions of the sensing sensor;

FIG. 4 is an exploded perspective view illustrating a lower surface side of a part of the sensing sensor;

FIG. 5A is a plan view illustrating a front surface of a wiring board;

FIG. 5B is a plan view illustrating a back surface of the wiring board;

FIG. 6A is a plan view illustrating a front surface of a crystal resonator;

FIG. 6B is a plan view illustrating a back surface of the crystal resonator;

FIG. 7 is a longitudinal cross-sectional side view of the sensing sensor;

FIG. 8 is a longitudinal cross-sectional side view enlarging the crystal resonator and a terminal portion of the sensing sensor;

FIG. 9 is a plan view of a rear side of the sensing sensor;

FIG. 10 is a perspective view illustrating a connector;

FIG. 11 is a cross-sectional view illustrating a connecting terminal;

FIG. 12 is a schematic configuration diagram of the sensing device;

FIG. 13 is an explanatory drawing explaining an operation of the sensing sensor of this disclosure; and

FIG. 14 is an explanatory drawing explaining an operation of the sensing sensor of this disclosure.

### DETAILED DESCRIPTION

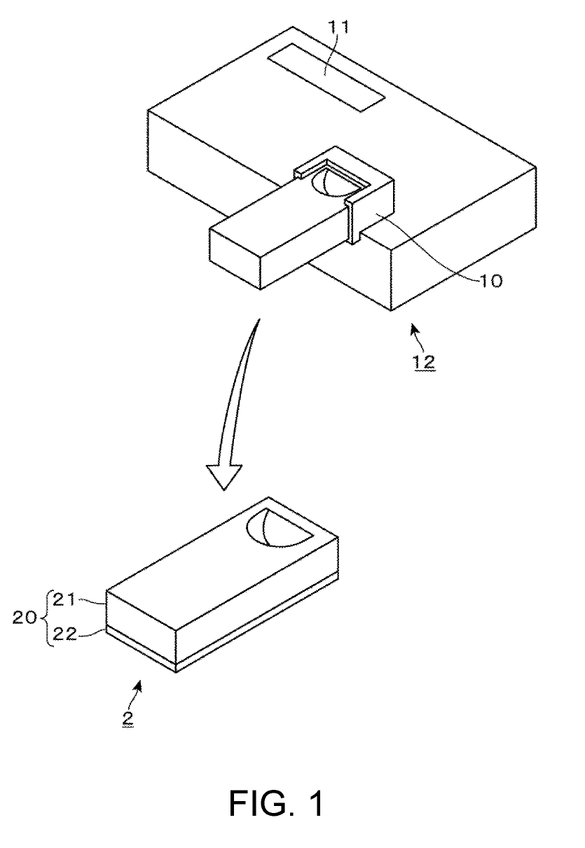

The following describes a sensing device using a sensing sensor according to an embodiment of the present disclosure. This sensing device uses a microfluidic chip. The sensing device can detect, for example, presence/absence of an antigen such as virus in sample solution obtained from nasal cavity swab of a human so as to determine whether the human has been infected with a virus or not with a microfluidic chip. As illustrated in an external perspective view in FIG. 1, the sensing device includes a main body 12 and a sensing sensor 2. The sensing sensor 2 is attachably/detachably connected to a connector 10 that is a holder holding the sensing sensor 2 disposed in the main body 12. The main body 12 includes, for example, a display 11 constituted by a liquid crystal display screen on a top surface. The display 11 displays, for example, output frequency of an oscillator circuit, which is disposed in the main body 12 and will be described later, a measurement result such as an amount of frequency transition, presence/absence of detected virus or a similar result.

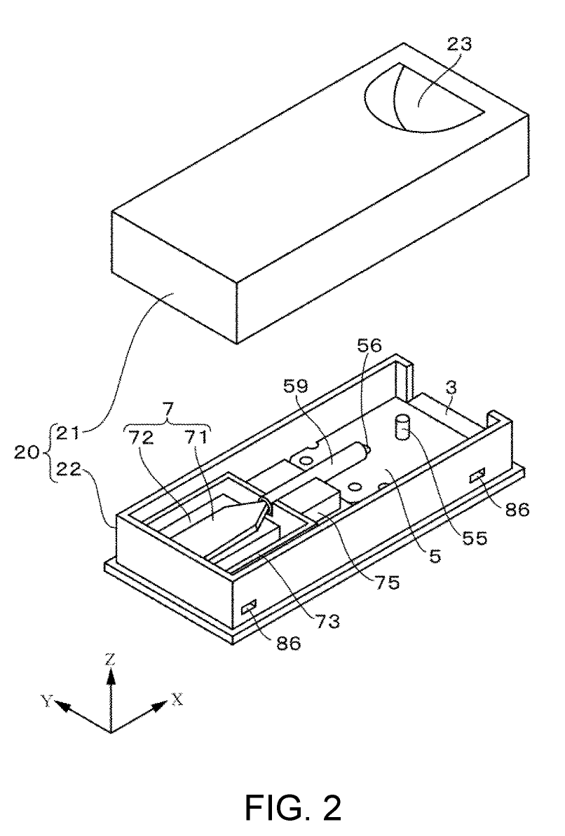

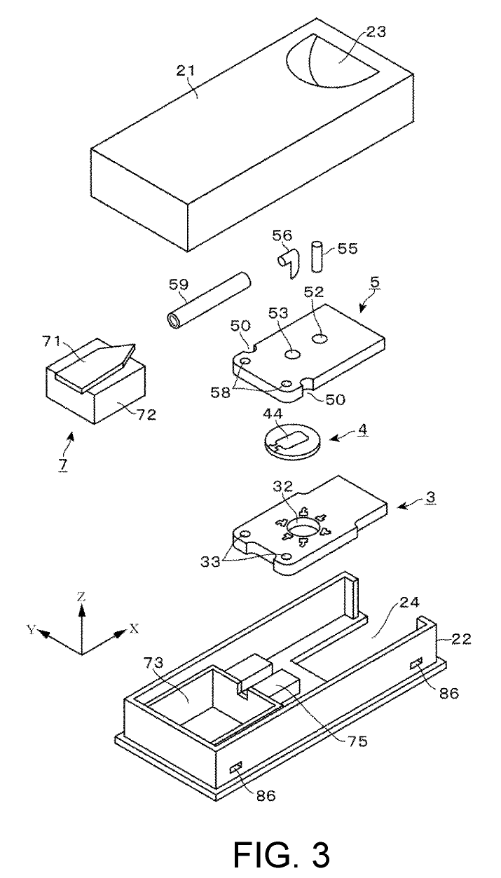

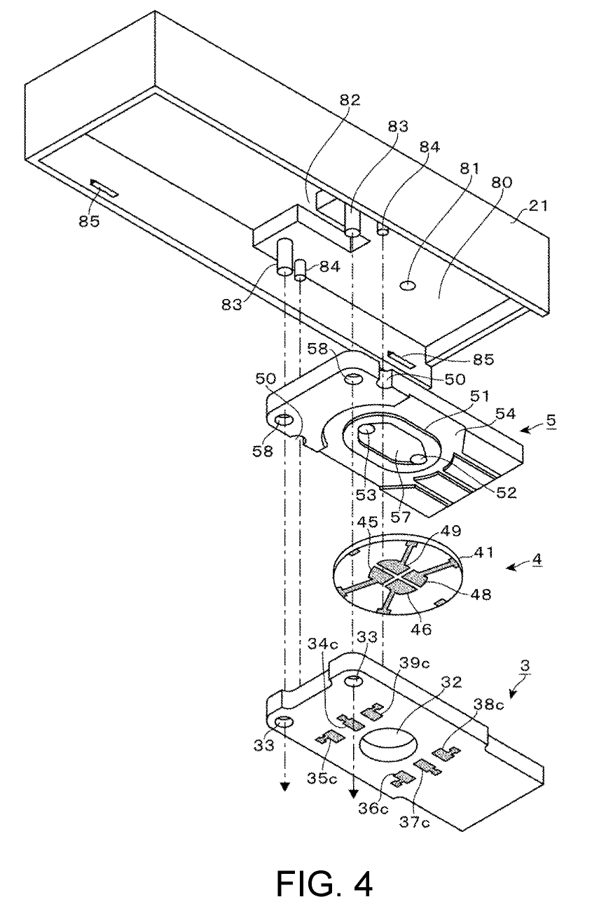

Next, the sensing sensor 2 will be described. FIG. 2 illustrates a perspective view of the sensing sensor 2, which is illustrated in FIG. 1, in a state where an upper-side case 21 is removed. FIG. 3 and FIG. 4 illustrate perspective views of a front side (top surface side) of respective members of the sensing sensor 2 and a back side (lower surface side) of some members, respectively. The sensing sensor 2 includes a case body 20 constituted of a lower-side case 22, which is a box shape with open upper side, and the upper-side case 21 with open lower side, which covers an outer wall of the lower-side case 22 from outside. Assuming that one end side of the lower-side case 22 is a rear side and the other end side is a front side, a part of a sidewall on the rear side of the lower-side case 22, for example, is cut out, and further, a bottom portion of the lower-side case 22 has a cutout 24 extending toward the front from an end portion on the rear side. Furthermore, outside surfaces of right and left sidewalls of the lower-side case 22 have grooves 86 to lock the upper-side case 21.

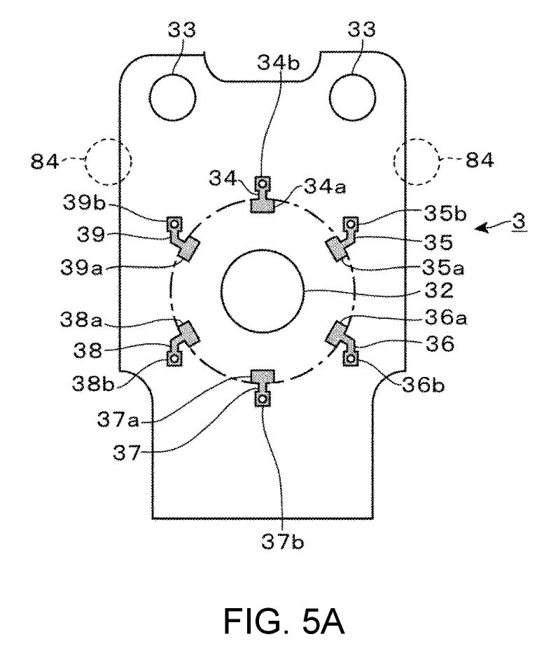

The lower-side case 22 internally includes a flat-plate-shaped wiring board 3. Describing also with reference to FIG. 5A and FIG. 5B, the wiring board 3 is constituted to be an approximately rectangular plate shape and a width of the front side part is wider than the rear side part. An approximate center of the wiring board 3 is formed with a penetration hole 32 and a position near the front of the wiring board 3 is formed with hole portions 33 arranged at two positions in a width direction to determine a horizontal position of the wiring board 3. A peripheral area of the penetration hole 32 on a front surface side of the wiring board 3 is formed with electrode terminals 34a to 39a at six positions keeping regular intervals in a circumferential direction with the penetration hole 32 as the center. To the electrode terminals 34a to 39a, respective conductive paths 34 to 39 each extending toward peripheral edges of the wiring board 3 are connected. The conductive paths 34 to 39 are each extended to a back surface side of the wiring board 3 via through-holes 34b to 39b. On the back surface side of the wiring board 3, the terminal portions 34c to 39c are arranged laterally by three on each of the front side and rear side of the penetration hole 32. The conductive paths 34 to 39, which are extended to the back surface side of the wiring board 3 via the through-holes 34b to 39b, are connected to the terminal portions 34c to 39c, respectively.

As described later, a crystal resonator 4 is secured to the position indicated by a chain line in FIG. 5A on the front surface side of the wiring board 3. As for the terminal portions 34c to 39c, the terminal portion 34c and the terminal portion 37c, the terminal portion 39c and the terminal portion 36c, and the terminal portion 35c and the terminal portion 38c are each symmetrically arranged viewing from the center of the crystal resonator 4. It is only necessary that the terminal portions 34c to 39c are arranged roughly symmetrically with respect to the center of the crystal resonator 4. The wiring board 3 is installed in the lower-side case 22 such that a region including the terminal portions 34c to 39c on the back surface side faces the outside from the cutout 24. Therefore, the cutout 24 is equivalent to a window portion.

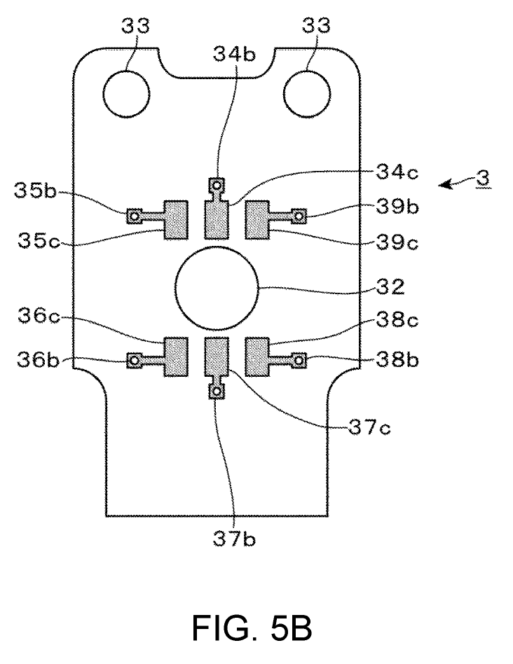

Next, a piezoelectric resonator, for example, the crystal resonator 4 will be described. As illustrated in FIG. 6A and FIG. 6B, the crystal resonator 4 includes, for example, an AT-cut circular plate-shaped crystal element 41. As illustrated in FIG. 6B, the back surface side of the crystal element 41 includes strip-shaped excitation electrodes 45, 46, 48 and 49 extending in the front-rear direction. The excitation electrodes 45, 46, 48 and 49 are disposed such that an electrode pair, which is the excitation electrodes 45 and 49 arranged to the right and left, and an electrode pair, which is the excitation electrodes 46 and 48 arranged to the right and left, are disposed to the front and rear, and the four excitation electrodes 45, 46, 48 and 49 are separate from one another via clearances.

As illustrated in FIG. 6A, the front surface side of the crystal element 41 includes an oval-shaped common electrode 44, which is made of Au, for example, extending in the front-rear direction. The common electrode 44 straddles over a region opposed to the four excitation electrodes 45, 46, 48 and 49 on the back surface side via the crystal element 41. Among four regions in the common electrode 44 opposed to the excitation electrodes 45, 46, 48 and 49 on the back surface side, a region on the right side in the front side viewing the common electrode 44 from the front surface side and opposed to the excitation electrode 45 is an adsorbing layer 42 formed into a strip shape. The adsorbing layer 42, which is made from antibody, to adsorb a sensing object, an antigen, extends in the front-rear direction. Similarly, a region in the common electrode 44 opposed to the excitation electrode 48 on the left side in the rear side is formed with the adsorbing layer 42 of a strip shape extending in the front-rear direction.

Surfaces of a region opposed to the excitation electrode 49 on the left side in the front side and a region opposed to the excitation electrode 46 on the right side in the rear side in the common electrode 44 do not include the adsorbing layer 42 and are exposed. In this crystal resonator 4, a vibrating region 101 interposed between the common electrode 44 and the excitation electrode 45 and a vibrating region 103 interposed between the common electrode 44 and the excitation electrode 48 include the adsorbing layer 42 on the surface of the common electrode 44 and become absorbing regions that adsorb the sensing object in the sample solution. A vibrating region 102 interposed between the common electrode 44 and the excitation electrode 49 and a vibrating region 104 interposed between the common electrode 44 and the excitation electrode 46 do not include the adsorbing layer 42 on the surface of the common electrode 44; thereby the sensing object is not adsorbed. Therefore, the vibrating region 102 and the vibrating region 104 are reference regions individually corresponding to the vibrating region 101 and the vibrating regions 103, the adsorbing regions, respectively.

The common electrode 44 is connected with one end of a wiring 44a that extends toward the peripheral edge on the front side of the crystal element 41. The other end side of the wiring 44a is extended to the back surface side via a side surface on the front side of the crystal element 41. An electrode 44b is formed at the peripheral edge in the front side on the back surface of the crystal element 41. The excitation electrodes 45, 46, 48 and 49 disposed on the back surface side of the crystal resonator 4 are connected with one ends of wirings 45a, 46a, 48a and 49a, respectively, that extend to the peripheral edge of the crystal resonator 4. The other end sides of the wirings 45a, 46a, 48a and 49a are each extended to the peripheral edge on the lower surface side of the crystal resonator 4, forming electrodes 45b, 46b, 48b and 49b. An electrode 47b formed in the rear on the back surface side of the crystal element 41 is a dummy electrode to align the height in the front and the rear of the crystal element 41 when the crystal resonator 4 is secured to the wiring board 3.

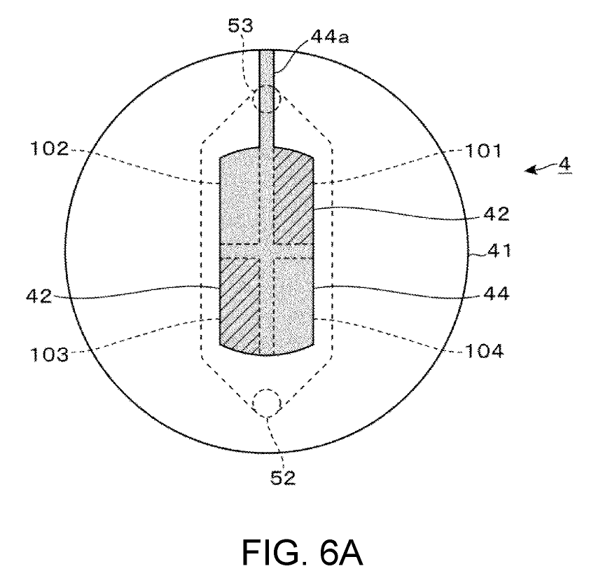

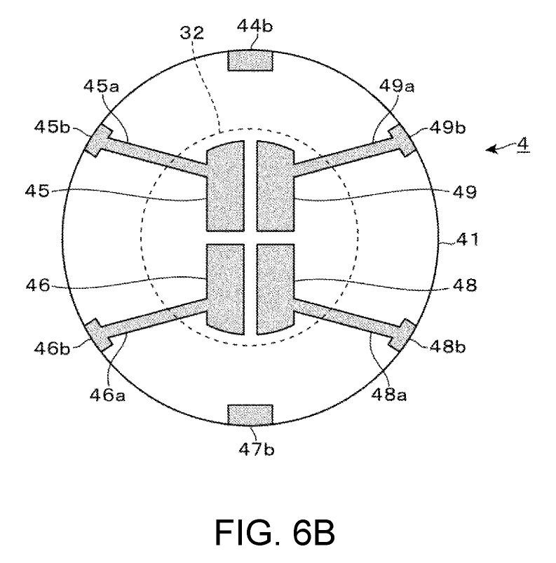

Describing also with reference to FIG. 7 and FIG. 8, the crystal resonator 4 is disposed such that the excitation electrodes 45, 46, 48 and 49 on the back surface side face the penetration hole 32 of the wiring board 3 as illustrated in FIG. 6B, and the electrodes 44b to 49b are connected to the electrode terminals 34a to 39a, respectively, with a conductive adhesive. According to the embodiment, the penetration hole 32 is equivalent to a depressed portion forming a clearance in a region facing the excitation electrodes 45, 46, 48 and 49 on the back surface side of the crystal resonator 4 on the wiring board 3.

Returning to FIG. 3 and FIG. 4, a top surface side of the wiring board 3 includes a channel forming member 5. The channel forming member 5 is constituted of, for example, a plate-shaped member of 2.0 mm thickness, and made of, for example, polydimethylsiloxane (PDMS) with an identical width to the front side portion of the wiring board 3. The position near the front of the channel forming member 5 includes hole portions 58 to position the channel forming member 5 at the positions corresponding to the hole portions 33 formed on the wiring board 3 such that the channel forming member 5 is penetrated in a thickness direction. Cutouts 50 are formed at positions near the front side in the channel forming member 5 on edges on the right and left in the rear of the hole portions 33.

The lower surface side of the channel forming member 5 is formed with a depressed portion 54 of an approximately circular shape so as to house the crystal resonator 4 as illustrated in FIG. 4. The depressed portion 54 includes a surrounding portion 51 that partitions and forms a channel 57 of the sample solution between with the surface of the crystal resonator 4 when the wiring board 3 is pressed to a side of the channel forming member 5. This surrounding portion 51 is constituted of a circular projecting portion having its outer edge formed to be an oval shape with its longitudinal direction toward the front-rear direction of the sensing sensor 2. The surrounding portion 51 is disposed to project from the depressed portion 54 by a thickness of 300 μm and the internal region of the surrounding portion 51 has a planar surface with a height identical to a height of the depressed portion 54. A width of the internal region of the surrounding portion 51 gradually increases from the rear side to the front side, then becomes a constant width at the middle, and gradually decreases toward the front side.

The channel forming member 5 includes a penetration hole 52 of 1.5 mm in diameter drilled to penetrate in the thickness direction and opening at a rear end of the internal region of the surrounding portion 51. The channel forming member 5 includes a penetration hole 53 as an effluent channel of 1.5 mm in diameter drilled to penetrate in the thickness direction and opening at a front end of the internal region of the surrounding portion 51. The hole portions 58 of the channel forming member 5 are arranged so as to align the hole portions 33 disposed in the wiring board 3. The surrounding portion 51 is disposed on the top surface of the crystal resonator 4. The common electrode 44 is housed in the center of the internal region of the surrounding portion 51 as illustrated in FIG. 6A. The lower surface side of the internal region of the surrounding portion 51 is covered with the crystal resonator 4. This region surrounded by the surrounding portion 51 and the crystal resonator 4 has the bottom surface constituted of the crystal resonator 4 to form the channel 57 of 300 μm in height with a ceiling surface and the bottom surface extending in parallel.

The penetration holes 52 and 53, as illustrated in FIG. 3, attachably/detachably include an inlet-side capillary member 55 and an outlet-side capillary member 56 each constituted of a porous member. The inlet-side capillary member 55, for example, is a columnar member and constituted of a chemical fiber bundle such as polyvinyl alcohol (PVA). The inlet-side capillary member 55 is disposed so as to cover the penetration hole 52. An upper end of the inlet-side capillary member 55 is disposed to expose to an injection port 23 formed in the upper-side case 21, which will be described later, and the lower end is disposed to enter into the channel 57. Similarly, the outlet-side capillary member 56 is constituted of a chemical fiber bundle such as polyvinyl alcohol (PVA) and is formed to be an L-shape by extending upward then bending to extend horizontally. The outlet-side capillary member 56 is disposed so as to cover the penetration hole 53 and a lower end of the outlet-side capillary member 56 is disposed to enter into the channel 57. Furthermore, the lower end of the outlet-side capillary member 56 is inclined to the front side from the rear side.

The other end side of the outlet-side capillary member 56 is connected to one end side of an effluent channel 59 constituted of, for example, a hydrophilic glass tube. The other end side of the effluent channel 59 is connected with, for example, a capillary sheet 71, which suctions liquid flown from the effluent channel 59, and an absorbing member 72, which absorbs liquid suctioned by the capillary sheet 71. A case body 73 to prevent a liquid leakage from the absorbing member 72 is disposed outside the absorbing member 72. Reference numeral 75 in the drawing is a supporting member that supports the effluent channel 59.

The upper-side case 21 is disposed so as to cover the wiring board 3, the channel forming member 5, and the absorbing member 72 from the upper side. The top surface side of the upper-side case 21 is formed with the injection port 23 inclined in a cone shape. As illustrated in FIG. 4, the back surface side of the upper-side case 21 includes a pressing portion 80 that regulates a height position of the channel forming member 5. The pressing portion 80 is constituted to be, for example, an approximately box shape, and the lower surface of the pressing portion 80 is brought into contact with an entire top surface of the channel forming member 5 when the upper-side case 21 is engaged and locked together with the lower-side case 22. The pressing portion 80 includes a penetration hole 81 penetrating to the injection port 23 at the position corresponding to the penetration hole 52. A cutout 82, which will be described later, to ensure an installation area for the outlet-side capillary member 56 is formed toward the front side from the position corresponding to the penetration hole 53. The pressing portion 80 includes fixing pillars 83. Regulating portions 84 that each regulates a height position of the wiring board 3 are disposed at positions near the outside in the rear side of the fixing pillars 83. Furthermore, inner surface sides of right and left sidewalls of the upper-side case 21 include claws 85 at the positions corresponding to the grooves 86 formed in the lower-side case 22.

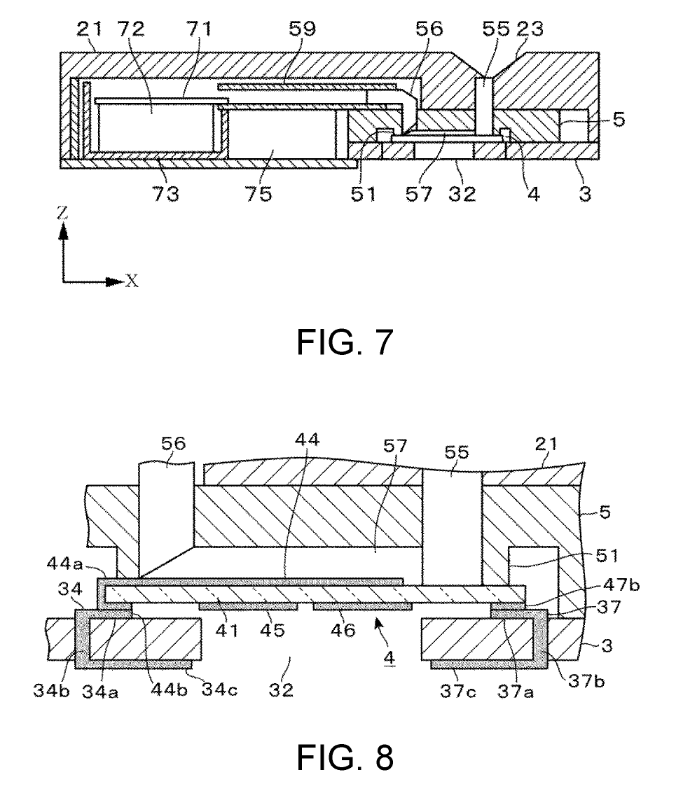

As illustrated in FIG. 9, when the wiring board 3 and the channel forming member 5 are stacked on the lower-side case 22, the peripheral edge portions of the wiring board 3 are faced to the cutouts 50 formed in the channel forming member 5. Furthermore, when the upper-side case 21 is covered, the fixing pillars 83 are inserted to the hole portions 33 and 58 formed in the wiring board 3 and the channel forming member 5, thus positioning the wiring board 3 and the channel forming member 5. The regulating portions 84 are fitted into the cutouts 50 of the channel forming member 5, and forward ends of the regulating portions 84 are positioned to be brought into contact with the peripheral edge portion on the front surface side of the wiring board 3. Furthermore, the claws 85 are engaged with the grooves 86 of the lower-side case 22 and secured.

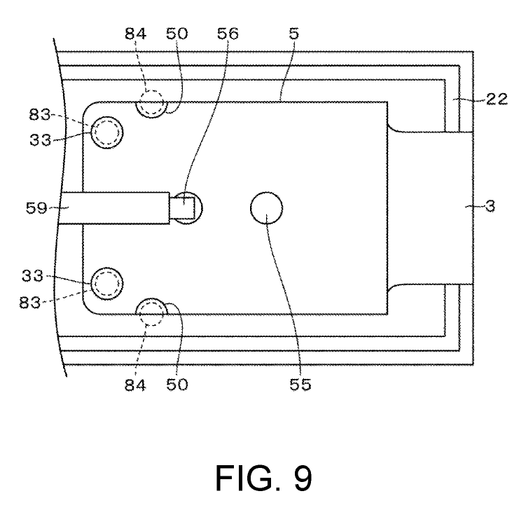

Next, the main body 12 will be described. The main body 12 is constituted of a chassis, and an end portion in the front side on the top surface includes the connector 10 to connect to the sensing sensor 2. As illustrated in FIG. 1 and FIG. 10, the connector 10 is constituted to be an approximately box shape having an opening in the front side. The connector 10 is configured such that the opening part is inserted with a rear side portion of the sensing sensor 2. A part of a ceiling portion of the connector 10 is formed with a cutout 13 which is faced to the injection port 23 when the sensing sensor 2 is inserted. The nearby portion of the cutout 13 in the ceiling portion of the connector 10 is configured to cover above the peripheral edge portion in the rear side of the inserted sensing sensor 2.

A bottom surface of the connector 10 includes two base portions 60 of rectangular shape extending in the width direction. The one base portion 60 is arranged behind the other. When the sensing sensor 2 is inserted into the connector 10, each of the base portions 60 enter the cutout 24 formed in the lower-side case 22 of the sensing sensor 2, and top surfaces of the base portions 60 are opposed to the lower surface of the wiring board 3 via a very narrow clearance. The top surfaces of the respective base portions 60 include three connecting terminals 6 arranged at regular intervals in the direction to which the base portions 60 extend. The connecting terminals 6 correspond to the respective six terminal portions 34c to 39c of the sensing sensor 2.

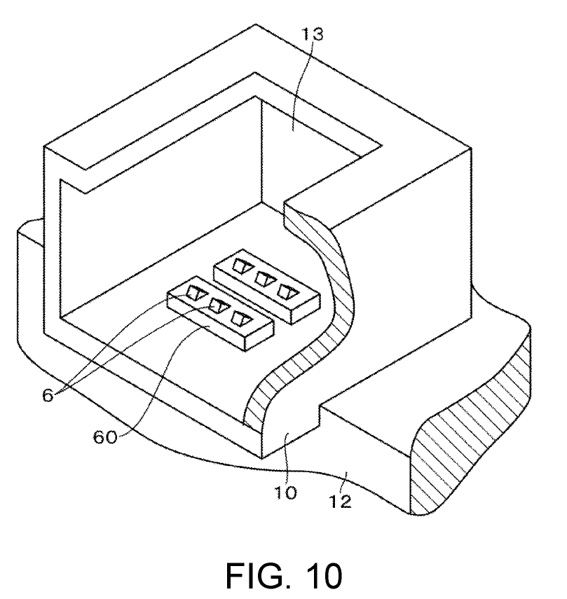

As illustrated in FIG. 11, the connecting terminal 6 is disposed such that a distal end projects upward from a hole portion 61 formed in the base portion 60, and constituted of a metal plate bent rearward on the upper side of the base portion 60. The distal end of the connecting terminal 6 is inserted from the upper side to this hole portion 61 and locked with an edge of a ceiling portion of the base portion 60. In view of this, the distal end of the connecting terminal 6 is always biased upward and when a bent portion 62 is pressed downward, the upward biasing force presses back. A height dimension of the projecting portion of the connecting terminal 6 from the base portion 60 is configured to be larger than a height dimension of a clearance from the top surface of the base portion 60 to the lower surface of the wiring board 3 when the sensing sensor 2 is inserted to the connector 10. Base end sides of the connecting terminals 6 are connected to respective conductive paths 64 formed in a wiring board 63 disposed inside the main body 12. The latter stage part of the conductive path 64 is connected with a switch portion 90 and an oscillator circuit 9 in this order.

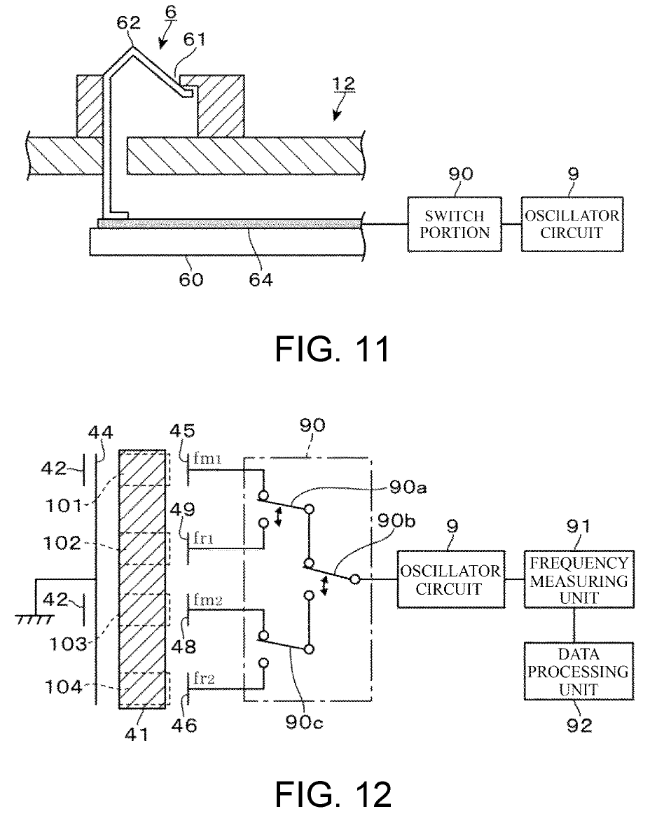

When the sensing sensor 2 is inserted to the connector 10, the connecting terminals 6 are pressed down in a state where the respective connecting terminals 6 are in contact with the corresponding terminal portions 34c to 39c on the sensing sensor 2 side and electrically connected. In view of this, the crystal resonator 4 is connected to the oscillator circuit 9 via the switch portion 90. Next, an overall configuration when the sensing sensor 2 is connected to the main body 12 will be described. As illustrated in FIG. 12, the switch portion 90 is constituted of three switches 90a to 90c. The switch portion 90 is configured to oscillate any one of the regions of the respective vibrating regions (the crystal element 41 in the respective regions in details) 101 and 102 interposed between the common electrode 44 and the excitation electrodes 49 and 45 and the respective vibrating regions 103 and 104 interposed between the common electrode 44 and the excitation electrodes 48 and 46, and to retrieve the oscillation output (frequency signal) in this one region to a side of the oscillator circuit 9.

Between the vibrating regions 101 and 102 and the oscillator circuit 9, the first switch 90a and the second switch 90b are disposed in this order from a side of the crystal resonator 4. The first switch 90a is configured to connect the oscillator circuit 9 to any one of the vibrating regions 101 and 102. The second switch 90b is switchably interposed between a connecting point on a side of these vibrating regions 101 and 102 and a connecting point on a side of the vibrating regions 103 and 104. Between the vibrating regions 103 and 104 and the second switch 90b, the third switch 90c configured to connect any one of the vibrating regions 103 and 104 with respect to the oscillator circuit 9 is disposed. A frequency measuring unit 91 is disposed at a latter stage of the oscillator circuit 9. A frequency measured by the frequency measuring unit 91 is input to a data processing unit 92.

The sensing device according to this disclosure sequentially retrieves time-series data of respective frequencies fm1, fr1, fm2 and fr2 in the respective vibrating regions 101 to 104 while switching the switch portion 90 at high speed. Then, the data processing unit 92 calculates each of a difference of oscillation frequency in the vibrating regions 101 and 102 Δf1 (Δf1=fm1−fr1) and a difference of oscillation frequency in the vibrating regions 103 and 104 Δf2 (Δf2=fm2−fr2) to calculate a summed value Δfsum of these differences Δf1 and Δf2 (Δfsum=Δf1+Af2). The sensing device according to this disclosure includes functions such as determining presence/absence of a sensing object on the basis of obtained calculation result (the summed value Δfsum) or displaying a density of a sensing object on the display 11 by reading the density corresponding to the calculation result from a calibration curve.

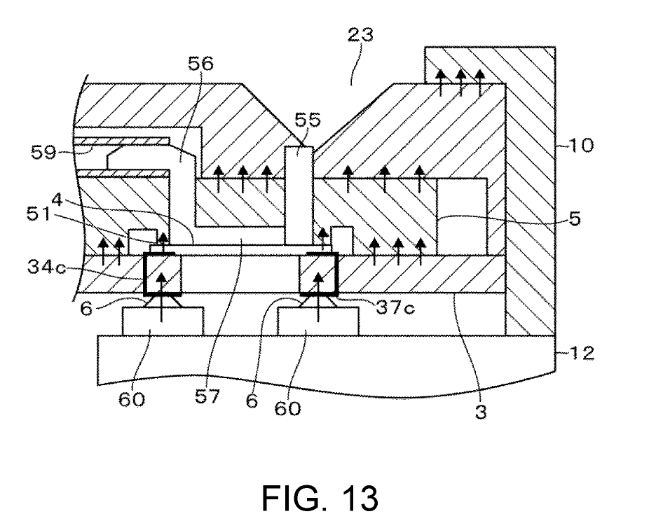

The above-described action of the embodiment will be described. When the sensing sensor 2 is connected to the connector 10, the connecting terminals 6 on the connector 10 side are each positioned below the corresponding terminal portions 34c to 39c on the sensing sensor 2 side. Since the connecting terminals 6 each have larger height dimensions than the clearance dimension between the top surface of the base portion 60 and the lower surface of the wiring board 3 when the sensing sensor 2 is inserted to the connector 10, the connecting terminals 6 are each pressed down by the terminal portions 34c to 39c on the sensing sensor 2 side. This causes the terminal portions 34c to 39c on the sensing sensor 2 side to be pressed up by a biasing force of the respective connecting terminals 6 returning upward and the wiring board 3 is pressed up as illustrated in FIG. 13. Since the terminal portions 34c to 39c are symmetrically disposed viewing from the center portion of the crystal resonator 4, the crystal resonator 4 is pressed up at symmetrical positions viewed from the center portion and pressed against the surrounding portion 51 of the channel forming member when the wiring board 3 is pressed up by the connecting terminals 6.

The force to the symmetrical positions viewed from the center portion causes the crystal resonator 4 to be pressed against the surrounding portion 51 with a further horizontal posture, thereby providing a further close contact with the surrounding portion 51. Therefore, the terminal portions 34c to 39c being symmetrically disposed viewed from the center portion of the crystal resonator 4 is included in a scope of rights because locating the center of the positions where the terminal portions 34c to 39c are disposed at the almost center portion of the crystal resonator 4 improves adhesion of the crystal resonator 4 and the surrounding portion 51.

As illustrated in FIG. 13, the channel forming member 5 is pressed up as the wiring board 3 is pressed up, and further, the upper-side case 21 is pressed up. However, the peripheral edge portion in the rear side of the upper-side case 21 is locked with the ceiling surface of the connector 10. Accordingly, the wiring board 3, the channel forming member 5, and the upper-side case 21 are brought into close contact with one another.

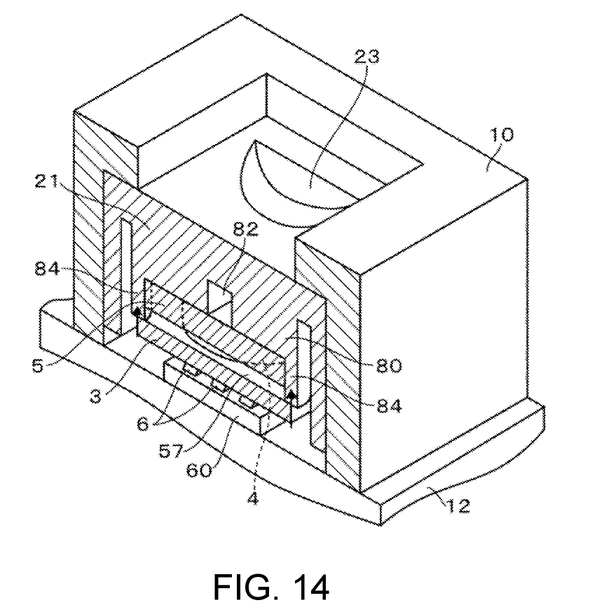

As illustrated in FIG. 14, the upper-side case 21 includes the regulating portion 84 that presses the peripheral edge portions of the wiring board 3. Therefore, the wiring board 3 is prevented from being excessively pressed against the channel forming member 5 when the wiring board 3 is pressed up, thus avoiding the channel 57 to be crushed.

The following describes a method for determining presence/absence of a sensing object in a sample solution by this sensing device. First, the sensing sensor is connected to the main body 12. A thinner, which is constituted of, for example, normal saline, and does not include a sensing object is dropped into the injection port 23 using an injector (not illustrated). The liquid is absorbed into the inlet-side capillary member 55 by capillary phenomenon, passes through inside this inlet-side capillary member 55 to flow into the channel 57, and then supplied to the surface on the rear side of the crystal resonator 4.

The surface of the crystal element 41 that constitutes the crystal resonator 4 is hydrophilic. Therefore, subsequent to liquid that has wet inside the channel 57 and spread to the channel 57, liquid in the inlet-side capillary member 55 is drawn to the surface of the crystal element 41 by surface tension. Thus, the liquid flows continuously from the injection port 23 to the channel 57. Since the vibrating regions 101 and 102 and the vibrating regions 103 and 104 are each disposed aligned in the proximity of the midstream of a supply channel, the liquid simultaneously flows at a constant speed on the surfaces of the vibrating regions 101 and 102 and simultaneously flows at a constant speed on the surfaces of the vibrating regions 103 and 104.

When the liquid on the surface of the crystal resonator 4 reaches the outlet-side capillary member 56, the liquid is absorbed into the outlet-side capillary member 56 by capillary phenomenon and flows inside this outlet-side capillary member 56 to exude to the effluent channel 59. The liquid inside the effluent channel 59 passes through inside this effluent channel 59 to the downstream side and reaches the capillary sheet 71.

Returning to the description of the buffer solution supply, the buffer solution dropped into the injection port 23 passes through inside the sensing sensor 2 as described above. When the buffer solution flowing through the channel 57 is supplied to the surface of the common electrode 44, these vibrating regions 101 and 102 and vibrating regions 103 and 104 equally receive an influence of hydraulic pressure since they are symmetrically formed viewed from the inlet side to the outlet side of the channel 57. This equally reduces the oscillation frequencies of both the vibrating regions 101 and 102 and equally reduces the oscillation frequencies of both the vibrating regions 103 and 104.

Next, a sample solution of equal amount to the buffer solution is supplied to the injection port 23. In view of this, a pressure applied to the buffer solution absorbed in the inlet-side capillary member 55 increases to flow this buffer solution again inside the effluent channel 59 to the downstream side and the sample solution is absorbed into the inlet-side capillary member 55. The absorbed sample solution flows in the channel 57 from the inlet-side capillary member 55 similar to the buffer solution and in the inside of the channel 57, the buffer solution is replaced by the sample solution.

Even in this time, the vibrating regions 101 and 102 and the vibrating regions 103 and 104 are symmetrically formed viewing in the front-rear direction of the channel 57. Therefore, these vibrating regions 101 and 102 and vibrating regions 103 and 104 equally receive the pressure change caused by the replacement of the liquid inside the channel 57 to cause the oscillation frequencies of the vibrating regions 101 and 102 to equally change with one another and the oscillation frequencies of the vibrating regions 103 and 104 to equally change with one another by this pressure change. In the case where a sensing object is included in the sample solution, this sensing object is absorbed into the adsorbing layers 42, which are on the vibrating regions 101 and 103, of the common electrode 44. Meanwhile, the sensing object is not adsorbed into the vibrating regions 102 and 104. Accordingly, the frequency decreases according to the absorption amount of the sensing object into the adsorbing layer 42, and Δfsum changes. Thus, the presence/absence of the sensing object can be determined on the basis of Δfsum change.

Thus, the sample solution is passed through the channel 57 formed inside the sensing sensor 2 to sense the sensing object. The channel 57 of the sensing sensor 2 is constituted by securing the crystal resonator 4 on the top surface of the wiring board 3, covering the upper-side case 21 from the upper side, and pressing the channel forming member 5 against the wiring board 3 by the pressing portion inside the upper-side case 21. Therefore, individual errors of design dimensions of the wiring board 3, the channel forming member 5, the upper-side case 21, and the lower-side case 22 may generate a gap between the wiring board 3 and the channel forming member 5 and leak the liquid flowing in the channel.

According to the above-described embodiment, the upward biasing force is provided to the connecting terminal 6 formed inside the connector 10 of the main body 12. Therefore, when the connector 10 is connected to the sensing sensor 2 and the connecting terminals 6 are pressed with the terminal portions 34c to 39c, the terminal portions 34c to 39c are pressed upward, thereby bringing the crystal resonator 4 into closer contact with the surrounding portion 51 disposed in the channel forming member 5. Therefore, a gap between the crystal resonator 4 and the surrounding portion 51 caused by individual errors of design dimensions is less likely to be generated and the leakage of the liquid flowing in the channel 57 is reduced. Furthermore, since the terminal portions 34c to 39c are symmetrically disposed viewing from the center portion of the crystal resonator 4, the crystal resonator 4 is pressed up by a force equal in a circumferential direction. Accordingly, the crystal resonator 4 is pressed against the surrounding portion 51 in a further horizontal posture, further improving the adhesion.

Since the height dimension of the channel 57 is small, the crystal resonator 4, which becomes the bottom surface of the channel 57, may be brought into contact with the ceiling of the channel 57 when the wiring board 3 is pressed up by the connecting terminals 6 and the channel 57 may be collapsed. However, the regulating portions 84 are disposed in the upper-side case 21 to hold the edge portions of the wiring board 3, thereby preventing the channel 57 from being collapsed.

Furthermore, according to the above-described sensing sensor 2, the wiring board 3 is exposed at the lower surface of the sensing sensor 2, and the crystal resonator 4 secured on the surface of the wiring board 3 may be damaged by colliding against the ceiling surface of the channel 57 by the wiring board 3 being pressed by an impact. Even with such a case, pressing the wiring board 3 by the regulating portion 84 of the upper-side case 21 prevents the collision of the crystal resonator 4 on the wiring board 3 against the channel forming member 5.

According to the above-described embodiment, the wirings 44a to 49a connected to the common electrode 44 and the excitation electrodes 45 to 49 of the crystal resonator 4 are extended to the lower surface of the wiring board 3 with through-holes 44b to 49b, thus forming terminal portions 44c to 49c on the lower surface side of the wiring board 3. In the case where the terminal portions 34c to 39c are formed on the front surface side of the wiring board 3, a region to dispose the crystal resonator 4 and a region to form the terminal portions 44c to 49c are necessary on one surface side of the wiring board 3. Since the one surface side of the wiring board 3 includes the channel forming member 5 so as to cover the region where the crystal resonator 4 is disposed, the region where the terminal portions 44c to 49c are formed is required to be disposed at a region outside the projection region of the channel forming member 5.

In the case where the terminal portions 44c to 49c are formed on the back surface side of the wiring board 3 and the crystal resonator 4 is formed on the other surface of the terminal portions 44c to 49c, the terminal portions 44c to 49c can be disposed in the projection region of the channel forming member 5. Therefore, the region not covered by the channel forming member 5 in the wiring board 3 can be narrowed, thus ensuring reducing the size of the wiring board 3 and reducing the size of the sensing sensor 2.

While this disclosure similarly provides the effect even with the sensing sensor 2 including a pair of adsorbing region and reference region in the crystal resonator 4, in the case where a plurality of pairs of the adsorbing regions and the reference regions are disposed in the crystal resonator 4 as described in the above-described embodiment, the region where the terminal portions 44c to 49c are formed is required to be wider because the number of the terminal portions 44c to 49c for connecting the common electrode 44 and the excitation electrodes 45 to 49 to the oscillator circuit 9 increases. Therefore, the wiring board 3 tends to increase in size by disposing the region to dispose the crystal resonator 4 and the region to form the terminal portions 44c to 49c on the one surface side of the wiring board 3; however, according to this disclosure, an increase in the size of the wiring board 3 can be inhibited, thereby ensuring inhibiting the increased size of the sensing sensor 2.

A sensing device according to the embodiment may include the sensing sensor described above and a main body including a holder holding the sensing sensor such that the injection port is faced to outside, an oscillator circuit that oscillates the piezoelectric resonator, and a connecting terminal that electrically connects the piezoelectric resonator to the oscillator circuit by pressing up the output terminal of the sensing sensor held by the holder from the other surface side.

The sensing sensor according to the embodiment secures the piezoelectric resonator including the adsorbing layer that adsorbs a sensing object to the excitation electrode on one surface side of the wiring board, covers the one surface side of the piezoelectric resonator with the channel forming member including the surrounding portion that forms the channel, forms the channel from one end side to the other end side on the one surface side of the piezoelectric resonator, and forms the terminal portion electrically connected to the excitation electrode on the other surface side of the wiring board. The connecting terminal connected to the oscillator circuit is pressed against the terminal portion to bring the piezoelectric resonator into close contact with the channel forming member and electrically connect the oscillator circuit to the excitation electrode. The wiring board being pressed by the connecting terminal covers a gap generated by individual error of each of the members constituting the sensing sensor because the crystal resonator is pressed against the sidewall of the channel formed in the channel forming member, thereby preventing leakage of a sample solution flowing in the channel. Furthermore, disposing the regulating portion that regulates the wiring board from the one surface side in the case body that houses the wiring board, the piezoelectric resonator, and the channel forming member prevents the channel from being crushed or the crystal resonator from colliding against the channel forming member when the wiring board is pressed from the other surface side.

The principles, preferred embodiment and mode of operation of the disclosure have been described in the foregoing specification. However, the disclosure which is intended to be protected is not to be construed as limited to the particular embodiments disclosed. Further, the embodiments described herein are to be regarded as illustrative rather than restrictive. Variations and changes may be made by others, and equivalents employed, without departing from the spirit of the disclosure. Accordingly, it is expressly intended that all such variations, changes and equivalents which fall within the spirit and scope of the disclosure as defined in the claims, be embraced thereby.

What is claimed is:

1. A sensing sensor comprising:
a wiring board including a depressed portion formed on one surface side, an output terminal formed on another surface side, and a conductive path connected to the output terminal and extended to the one surface side;
a piezoelectric resonator configured by disposing an excitation electrode electrically connected to the conductive path in a piezoelectric piece, the piezoelectric resonator being formed with an adsorbing layer adsorbing a sensing object in a sample solution on the one surface side, the piezoelectric resonator being secured to the wiring board in a state where the depressed portion is covered such that a vibrating region is opposed to the depressed portion;
a channel forming member disposed to cover a region on the one surface side of the wiring board including the piezoelectric resonator, the channel forming member including a surrounding portion forming a channel that passes a supply liquid from one end side to another end side on the one surface side of the piezoelectric resonator between the piezoelectric resonator and the channel forming member;
a case body housing the wiring board, the piezoelectric resonator, and the channel forming member, the case body including a window faced to a region including the output terminal on the another surface side of the wiring board and an injection port supplying a supply liquid to the one end side of the channel; and
a regulating portion disposed in the case body, the regulating portion regulating the wiring board from the one surface side when the wiring board is pressed from the another surface side, wherein
the piezoelectric resonator is pressed against the surrounding portion by the output terminal being pressed from the another surface side.

2. The sensing sensor according to claim 1, wherein
a plurality of the output terminals are disposed, and one output terminal and another output terminal of the plurality of output terminals are symmetrically disposed viewing from a center portion of the piezoelectric resonator.

3. The sensing sensor according to claim 1, wherein
the output terminal is disposed in a projection region of the channel forming member.

4. The sensing sensor according to claim 1, wherein
the excitation electrode includes:
a common electrode formed on the one surface side of the piezoelectric piece;
a first opposing electrode on the another surface side of the piezoelectric piece individually formed to be opposed to a first adsorbing region where the adsorbing layer is formed on a surface of the common electrode and a first reference region disposed in a direction intersecting with a flow direction of the fluid viewing from the first adsorbing region without an adsorbing layer on a surface of the common electrode; and
a second opposing electrode on the another surface side of the piezoelectric piece individually formed to be opposed to a second adsorbing region formed with the adsorbing layer on a surface of the common electrode in a position separating to the one side or the other side viewing from the first reference region and a second reference region disposed in a direction intersecting with a flow direction of the fluid viewing from this second adsorbing region without the adsorbing layer on a surface of the common electrode.

5. A sensing device comprising:
the sensing sensor according to claim 1; and
a main body including a holder holding the sensing sensor such that the injection port is faced to outside, an oscillator circuit that oscillates the piezoelectric resonator, and a connecting terminal that electrically connects the piezoelectric resonator to the oscillator circuit by pressing up the output terminal of the sensing sensor held by the holder from the another surface side.
